(19) United States
(12) Patent Application Publication
(10) Pub. No.: US 2025/0222173 A1
(43) Pub. Date: Jul. 10, 2025

(54) METHOD FOR PRODUCING OCTACALCIUM PHOSPHATE MOLDED ARTICLE COMPOUNDED WITH BIOCOMPATIBLE POLYMER

(71) Applicant: NATIONAL INSTITUTE OF ADVANCED INDUSTRIAL SCIENCE AND TECHNOLOGY, Tokyo (JP)

(72) Inventor: Yuki SUGIURA, Kagawa (JP)

(21) Appl. No.: 18/855,176

(22) PCT Filed: Apr. 5, 2023
(86) PCT No.: PCT/JP2023/014061

§ 371 Date: Oct. 8, 2024

(30) Foreign Application Priority Data
Apr. 11, 2022 (JP) 2022-065131

(51) Int. Cl. A61L 27/46; A61L 27/16; A61L 27/20; A61L 27/22

(52) U.S. Cl. CPC A61L 27/46; A61L 27/16; A61L 27/20; A61L 27/222; A61L 2400/12; A61L 2430/02

(57) ABSTRACT

The present invention aims to provide a method of producing an octacalcium phosphate molded body that is a composite containing a biocompatible polymer. A method of producing an octacalcium phosphate molded body that is a composite containing a biocompatible polymer, the method including the steps of mixing a compound containing a calcium ion and a compound containing a phosphate ion with a solution containing a biocompatible polymer, to prepare a mixed slime; and filling the mixed slime into a mold, to react the calcium ion with the phosphate ion.

### TECHNICAL FIELD

The present invention relates to a method of producing an octacalcium phosphate (OCP) molded body that is a composite containing a biocompatible polymer.

### BACKGROUND ART

Since calcium phosphate promotes bone induction and tissue regeneration, it is used as a material for reconstructing or regenerating a bone defect site resulting from an injure or a disease (bone substituting material). Calcium phosphate to be implanted in vivo is designed based on the premise that bone replacement occurs through the action of osteoclasts, and that osteoblasts act with osteoclasts in a coordinated manner to allow appropriate functioning of the bone remodeling process (bone metabolism). On the other hand, in Japan, there are several million people suffering from rheumatoid arthritis or osteoporosis. In such cases, the bone remodeling process may not appropriately function, and hence the application of bone-filling materials requires careful consideration at present.

In a known technique for increasing the functionality of a bone-filling material, a calcium phosphate-based compound is used as a base material, and the compound is allowed to support an agent. By allowing the compound to support an agent suitable for the purpose, effective action of the agent can be achieved even in cases where application of a current bone-filling material is difficult. For example, Patent Document 1 discloses a technique related to calcium phosphate supporting an agent such as indomethacin, insulin, or aspirin.

Patent document 2 discloses a biological hard-tissue-like hardened body that can be used for drug delivery systems, the hardened body comprising: a biopolymer such as collagen or hyaluronic acid; and a calcium phosphate-based compound. Composites containing these biopolymers improve the hard and brittle properties characteristic to ceramics and enable achievement of flexibility. Patent document 3 discloses a bone regeneration material comprising a composite of OCP granules and gelatin.

### PRIOR ART DOCUMENTS

### Patent Documents

[Patent Document 1] JP-A H6-228011
[Patent Document 2] JP-A 2003-93496
[Patent Document 3] WO 2020/071497

### SUMMARY OF THE INVENTION

### Problems to be Solved by the Invention

“Calcium phosphate” does not mean a single compound, and a plurality of kinds of calcium phosphates are known. Known examples of calcium phosphates used in the medical field, including those used as bone-filling materials, include calcium hydrogen phosphate dihydrate (DCPD), octacalcium phosphate (OCP), hydroxyapatite (HAp), carbonate apatite (CO₃Ap), α-tricalcium phosphate (α-TCP), and β-tricalcium phosphate (β-TCP). In particular, OCP is known to have excellent bone regenerative properties.

Calcium phosphate molded bodies can be prepared by dissolution-precipitation reaction. More specifically, a compound containing a calcium ion is reacted in a weakly basic phosphoric acid solution to induce dissolution-precipitation reaction, to prepare a calcium phosphate molded body. However, this method has the problem that preparation of a calcium phosphate molded body supporting an agent is difficult. The agent is supported in a calcium phosphate crystal, but, since sodium ions and ammonium ions as cations in the buffer solution used for the dissolution-precipitation reaction strongly inhibit the supporting of the agent, it is difficult for the dissolution-precipitation reaction to produce a molded body that sufficiently maintains a shape and strength.

In view of such a problem, the present inventors intensively studied to discover that a molded body that sufficiently maintains a shape and strength and that is a composite containing a biocompatible polymer can be prepared by kneading the biocompatible polymer with a compound containing a calcium ion and the like in advance, molding the kneaded product, and then reacting the calcium ion with a phosphate ion to synthesize OCP, thereby reaching the present invention. Based on such a discovery, the present invention aims to provide a method of producing an OCP molded body that is a composite containing a biocompatible polymer.

### Means for Solving the Problems

One aspect of the present invention that can solve the above problem is as follows.

[1] A method of producing an OCP molded body that is a composite containing a biocompatible polymer, the method comprising the steps of:
mixing a compound containing a calcium ion and a compound containing a phosphate ion with a solution containing a biocompatible polymer, to prepare a mixed slime; and
filling the mixed slime into a mold, to react the calcium ion with the phosphate ion.

[2] A method of producing an octacalcium phosphate molded body that is a composite containing a biocompatible polymer, the method comprising the steps of:
mixing a compound containing a calcium ion with a solution containing a biocompatible polymer, to prepare a mixed slime;
adding a compound containing a phosphate ion to the mixed slime, and mixing the resulting mixture; and
filling the resulting mixed slime into a mold, to react the calcium ion with the phosphate ion.

[3] A method of producing an OCP molded body that is a composite containing a biocompatible polymer, the method comprising the steps of:
mixing a compound containing a calcium ion with a solution containing a biocompatible polymer, to prepare a mixed slime;
filling the mixed slime into a mold, and then freezing the mixed slime; and
immersing the molded body obtained by the freezing, in a solution of a compound containing a phosphate ion, to react the calcium ion with the phosphate ion.

[4] The method of producing an OCP molded body that is a composite containing a biocompatible polymer according to any one of [1] to [3], wherein the compound containing a calcium ion is one or more selected from the group consisting of calcium dihydrogen phosphate hydrate, anhydrous calcium dihydrogen phosphate, calcium hydrogen phosphate dihydrate, anhydrous calcium hydrogen phosphate, hydroxyapatite, carbonate apatite, sodium-substituted hydroxyapatite, α-tricalcium phosphate, β-tricalcium phosphate, and calcium carbonate.

[5] The method of producing an OCP molded body that is a composite containing a biocompatible polymer according to any one of [1] to [3], wherein the compound containing a phosphate ion is one or more selected from the group consisting of sodium dihydrogen phosphate, disodium hydrogen phosphate, trisodium phosphate, potassium dihydrogen phosphate, dipotassium hydrogen phosphate, tripotassium phosphate, ammonium dihydrogen phosphate, diammonium hydrogen phosphate, triammonium phosphate, and ammonium magnesium phosphate.

[6] The method of producing an OCP molded body that is a composite containing a biocompatible polymer according to any one of [1] to [4], wherein the biocompatible polymer is one or more selected from the group consisting of alginate, gelatin, hyaluronic acid, carboxylic acid-modified cellulose, sodium polyacrylate acid, polyethylene glycol, pullulan, phosphate modified pullulan, polyglutamic acid, fibrin, chitin, chitosan, and gellan gum.

### Effect of the Invention

The present invention can provide a method of producing an OCP molded body that is a composite containing a biocompatible polymer. OCP molded body that is a composite containing a biocompatible polymer, obtained by the present invention produces an excellent effect since it sufficiently maintains a shape and strength.

### BRIEF DESCRIPTION OF THE DRAWINGS

FIG. 1 shows a photograph of the molded body prepared in Example 1 (drawing-substituting photograph).

FIG. 2 shows a diagram illustrating the result of XRD analysis of the molded body prepared in Example 1.

FIG. 3 shows SEM photographs of the molded body prepared in Example 1 (drawing-substituting photographs).

FIG. 4 shows photographs of the molded body prepared in Example 2 (drawing-substituting photograph).

FIG. 5 shows a diagram illustrating the result of XRD analysis of each molded body prepared in Example 2.

FIG. 6 shows the result of measurement of the DTS strength of each molded body prepared in Example 2.

FIG. 7 shows a diagram illustrating a stress-strain curve of each molded body prepared in Example 2.

FIG. 8 shows SEM photographs of a molded body prepared in Example 2 (drawing-substituting photographs).

FIG. 9 shows a photograph of the molded body prepared in Examples 3 and 4 (drawing-substituting photograph).

FIG. 10 shows a diagram illustrating the result of XRD analysis of the molded body prepared in Examples 3 and 4.

FIG. 11 shows the result of measurement of the DTS strength of the molded body prepared in Examples 3 and 4.

FIG. 12 shows a diagram illustrating a stress-strain curve of the molded body prepared in Examples 3 and 4.

FIG. 13 shows a photograph of the molded body prepared in Example 5 (drawing-substituting photograph).

FIG. 14 shows the result of measurement of the DTS strength of the molded body prepared in Example 5.

FIG. 15 shows a diagram illustrating a stress-strain curve of the molded body prepared in Example 5.

FIG. 16 shows a diagram illustrating the result of XRD analysis of the molded body prepared in Example 5.

FIG. 17 shows a photograph of the molded body prepared in Example 6 (drawing-substituting photograph).

FIG. 18 shows a diagram illustrating the result of XRD analysis of the molded body prepared in Example 6.

FIG. 19 shows the result of measurement of the DTS strength of the molded body prepared in Example 6.

FIG. 20 shows photographs of the molded body prepared in Example 7 (drawing-substituting photographs).

FIG. 21 shows a diagram illustrating the result of XRD analysis of the molded body prepared in Example 7.

FIG. 22 shows SEM photographs of the molded body prepared in Example 7 (drawing-substituting photographs).

FIG. 23 shows a photograph of the molded body prepared in Example 8 (drawing-substituting photograph).

FIG. 24 shows a CT image of the molded body prepared in Example 8 (drawing-substituting photograph).

FIG. 25 shows a diagram illustrating the result of XRD analysis of the molded body prepared in Example 8.

### MODE FOR CARRYING OUT THE INVENTION

The present invention is described below in detail. The description of the following matters is an example (representative example) of embodiments of the present invention. The present invention is not limited to these contents, and may be carried out with various modifications within the scope of its spirit.

The molded body obtained by an embodiment of the present invention is a molded body that is a composite containing a biocompatible polymer, and that contains octacalcium phosphate (OCP) as a major component. The molded body can contribute mainly to the bone remodeling process. By reacting calcium ions with phosphate ions in the presence of a biocompatible polymer, a molded body that is a composite containing a biocompatible polymer can be obtained in the course of the crystal growth of octacalcium phosphate (OCP). In the composite, crystals are in a state where they are intertwining and fused to each other. Therefore, the shape of the molded body can be maintained as a whole, and the shape can be retained to an extent at which disintegration does not occur even in cases where the molded body is immersed in a solution containing distilled water, ethanol, or the like.

### [1] First Embodiment

The first embodiment is a method of producing an octacalcium phosphate molded body that is a composite containing a biocompatible polymer, the method comprising the steps of:
mixing a compound containing a calcium ion and a compound containing a phosphate ion with a solution containing a biocompatible polymer, to prepare a mixed slime; and
filling the mixed slime into a mold, to react the calcium ion with the phosphate ion.

In the first embodiment, calcium phosphate (excluding octacalcium phosphate) may be used as the compound containing a calcium ion. Examples of the calcium phosphate include calcium dihydrogen phosphate hydrate, anhydrous calcium dihydrogen phosphate, calcium hydrogen phosphate dihydrate, anhydrous calcium hydrogen phosphate, hydroxyapatite, carbonate apatite, sodium-substituted hydroxyapatite, α-tricalcium phosphate, and β-tricalcium phosphate. These may be used either individually or as a mixture containing two or more thereof at any ratios.

Besides the calcium phosphates described above, examples of the compound containing a calcium ion that may be used include inorganic calcium salts and organic calcium salts. Examples of the inorganic calcium salts include calcium carbonate, calcium hydroxide, calcium oxide, calcium chloride, calcium fluoride, calcium bromide, calcium iodide, calcium phosphide, calcium sulfate, calcium sulfate hemihydrate, calcium sulfate dihydrate, anhydrous calcium oxalate, calcium oxalate monohydrate, calcium oxalate dihydrate, calcium oxalate trihydrate, calcium sulfite, calcium silicate, calcium pyrophosphate, calcium tungstate, and calcium molybdate. Examples of the organic calcium salts include calcium acetate, calcium succinate, calcium citrate, calcium malate, calcium thiomalate, calcium benzoate, calcium lactate, and calcium stearate. These may be used either individually or as a mixture containing two or more thereof at any ratios.

In the first embodiment, examples of the compound containing a phosphate ion include sodium dihydrogen phosphate, disodium hydrogen phosphate, trisodium phosphate, potassium dihydrogen phosphate, dipotassium hydrogen phosphate, tripotassium phosphate, ammonium dihydrogen phosphate, diammonium hydrogen phosphate, triammonium phosphate, ammonium magnesium phosphate, and magnesium phosphate. Any one or more of these may be used.

In general, a biocompatible polymer is a material having the property of being not rejected and not recognized as a foreign substance by the living body after the implantation, and/or having the property of being compatible in vivo. The material is intended to be absorbed into the living body over time. For therapeutic purposes, the biocompatible polymer can be an agent. The biocompatible polymer is preferably compatible with the living body from the viewpoint of, for example, appropriate solubility in vivo and formation of a bond with a calcium salt. Since the biocompatible polymer needs to form a composite with a molded body containing octacalcium phosphate as a major component, the biocompatible polymer is preferably capable of having flexibility and toughness so as to improve formability and mechanical properties of the molded body.

In the present description, the polymer in the biocompatible polymer means a molecule having a molecular weight of more than 400, and having a structure composed of a large number of repeat units that are obtained substantially or conceptually from molecules having a small molecular weight. Thus, from the viewpoint of the polymerization state and the ordered-structure state of the polymer, the polymer includes those classified as linear polymers, star-shaped polymers, comb-shaped polymers, brush-shaped polymers, two-dimensional polymers, three-dimensional polymers, and the like. Side chains and repeating units of these polymers may contain other structures such as rotaxane structures.

A variety of materials may be used as the biocompatible polymer, and the polymer to be included in the composite is not limited. Examples of the polymer that may be used include alginate, gelatin, collagen, actin, fibrin, starch, dextrin, amylose, amylopectin, pectin, glycogen, curdlan, paramylon, agarose, carrageenan, heparin, xyloglucan, glucomannan, levan, fructan, pullulan, phosphorylate modified pullulan, fibroin, cellulose, chitosan, chitin, polyglycolic acid, polylactic acid, polyglycolic acid-polylactic acid copolymers, poly-L-lactic acid, melanin, hyaluronic acid, gellan gum, mastic gum, carboxylic acid-modified cellulose, lignin, casein, polyglutamic acid, sodium polyacrylate acid, and polyethylene glycol. One or more of these may be used.

In the present description, the solution containing a biocompatible polymer may be prepared by dissolving the biocompatible polymer in a solvent. The solvent is not limited, and is usually in the form of water or an aqueous solution. However, other solvents may also be used. In cases where solvents other than water are used, these solvents need to be liquids under the temperature/pressure conditions during the reaction. Examples of the solvent include monohydric alcohols such as primary alcohols including methanol, ethanol, propan-1-ol, butan-1-ol, pentan-1-ol, hexan-1-ol, heptan-1-ol, octan-1-ol, nonan-1-ol, and decan-1-ol, secondary alcohols including 2-propanol (isopropyl alcohol), butan-2-ol, pentan-2-ol, hexan-2-ol, and cyclohexanol, and tertiary alcohols including tert-butyl alcohol, 2-methylbutan-2-ol, 2-methylpentan-2-ol, 2-methylhexan-2-ol, 3-methylpentan-3-ol, and 3-methyloctan-3-ol; dihydric alcohols such as ethylene glycol and diethylene glycol; trihydric alcohols such as glycerin; aromatic alcohols such as phenol; polyethers such as polyethylene glycol (PEG) and polypropylene glycol (PPG); fatty acids such as acetic acid, valeric acid, caproic acid, lauric acid, palmitic acid, stearic acid, oleic acid, and linoleic acid; alkanes such as pentane, butane, hexane, heptane, and octane; esters such as ethyl acetate, methyl butyrate, methyl salicylate, ethyl formate, ethyl butyrate, ethyl caproate, octyl acetate, and dibutyl phthalate; compounds called cycloalkanes or bicycloalkanes such as cyclopentane, cyclohexane, and decalin; ketones such as acetone, methyl ethyl ketone, and diethyl ketone; aldehydes such as formaldehyde, acetaldehyde, propionaldehyde, butanal, pentanal, hexanal, and vanillin; and amine compounds such as aminomethane, aminoethane, ethylenediamine, triethylamine, and aniline. These may be used either individually or as a mixture of two or more thereof.

In a first embodiment, a compound containing a calcium ion and a compound containing a phosphate ion are mixed with a solution containing a biocompatible polymer, to prepare a mixed slime. The mixing method is not limited. For example, the solution containing a biocompatible polymer may be added dropwise to a mixed powder of the compound containing a calcium ion and the compound containing a phosphate ion, and the resulting mixture may be thoroughly mixed using a spatula or the like, to prepare the mixed slime. The mixing ratio is preferably 200:1 to 1:1 in terms of the weight ratio between the mixed powder of the compound containing a calcium ion and the compound containing a phosphate ion, and the biocompatible polymer. The mixing ratio is more preferably 100:1 to 2:1, still more preferably 50:1 to 3:1, especially preferably 20:1 to 5:1.

Subsequently, the resulting mixed slime is filled into a mold, and then cured after sealing the mold. The curing temperature and the curing time may be set appropriately. The curing temperature may be, for example, 5 to 95° C., or may be preferably 15 to 80° C. The curing time may be, for example, 2 to 96 hours, or may be preferably 6 to 72 hours. During the curing process (the process of reaction between the calcium ion and the phosphate ion), precipitation of calcium phosphate crystals as a composite containing the biocompatible polymer occurs. The cured product is dried using a dryer or the like, and then a molded body is removed from the mold. In cases where an unreacted component remains after the removal, the unreacted component is preferably removed by washing with distilled water or the like. By this, an octacalcium phosphate (OCP) molded body that is a composite containing the biocompatible polymer can be produced.

### [2] Second Embodiment

A second embodiment is a method of producing an OCP molded body that is a composite containing a biocompatible polymer, the method comprising the steps of:
mixing a compound containing a calcium ion with a solution containing a biocompatible polymer, to prepare a mixed slime;
adding a compound containing a phosphate ion to the mixed slime, and mixing the resulting mixture; and
filling the resulting mixed slime into a mold, to react the calcium ion with the phosphate ion.

In the second embodiment, examples of the compound containing a calcium ion that may be used include inorganic calcium salts and organic calcium salts. Examples of the inorganic calcium salts include calcium carbonate, calcium hydroxide, calcium oxide, calcium chloride, calcium fluoride, calcium bromide, calcium iodide, calcium phosphide, calcium sulfate, calcium sulfate hemihydrate, calcium sulfate dihydrate, anhydrous calcium oxalate, calcium oxalate monohydrate, calcium oxalate dihydrate, calcium oxalate trihydrate, calcium sulfite, calcium silicate, calcium pyrophosphate, calcium tungstate, and calcium molybdate. Examples of the organic calcium salts include calcium acetate, calcium succinate, calcium citrate, calcium malate, calcium thiomalate, calcium benzoate, calcium lactate, and calcium stearate. These may be used either individually or as a mixture containing two or more thereof at any ratios.

Besides the inorganic calcium salts and the organic calcium salts described above, calcium phosphate (excluding OCP) may be used as the compound containing a calcium ion. Examples of the calcium phosphate include calcium dihydrogen phosphate hydrate, anhydrous calcium dihydrogen phosphate, calcium hydrogen phosphate dihydrate, anhydrous calcium hydrogen phosphate, hydroxyapatite, carbonate apatite, sodium-substituted hydroxyapatite, α-tricalcium phosphate, and β-tricalcium phosphate. These may be used either individually or as a mixture containing two or more thereof at any ratios.

As the solution of a compound containing a phosphate ion, a solution containing an inorganic salt of phosphoric acid may be used. Examples of the inorganic salt of phosphoric acid include sodium dihydrogen phosphate, disodium hydrogen phosphate, trisodium phosphate, potassium dihydrogen phosphate, dipotassium hydrogen phosphate, tripotassium phosphate, ammonium dihydrogen phosphate, diammonium hydrogen phosphate, triammonium phosphate, and ammonium magnesium phosphate. These may be used either individually or as a mixture containing two or more thereof at any ratios.

In the second embodiment, a compound containing a calcium ion is mixed with a solution containing a biocompatible polymer. The mixing ratio is preferably 200:1 to 1:1 in terms of the weight ratio between the compound containing a calcium ion, and the biocompatible polymer. The mixing ratio is more preferably 100:1 to 2:1, still more preferably 50:1 to 3:1, especially preferably 20:1 to 5:1. The mixing method is not limited. For example, the solution containing a biocompatible polymer is added dropwise to the compound containing a calcium ion (powder), and the resulting mixture is thoroughly mixed using a spatula or the like. For example, a solution containing a biocompatible polymer obtained by dissolving the biocompatible polymer in a solvent is mixed with a raw material calcium carbonate powder, to prepare a mixed slime.

Subsequently, a solution of a compound containing a phosphate ion is mixed with the mixed slime. This allows, for example, the calcium carbonate contained in the mixed slime to react with the phosphoric acid in the solution, to form OCP. During the reaction, foaming may occur vigorously to cause partial curing, but it is preferred to continue the mixing until the foaming reaction settles down. In this case, the ratio of each compound may be adjusted such that the calcium ion reacts with the phosphate ion to form OCP.

Subsequently, the resulting mixed slime is filled into a mold, and then cured after sealing the mold. The curing temperature and the curing time may be set appropriately. The curing temperature may be, for example, 35 to 50° C., and the curing time may be, for example, 10 minutes to 96 hours. During the curing process (the process of reaction between the calcium ion and the phosphate ion), precipitation of OCP crystals containing the biocompatible polymer occurs. The cured product is dried using a dryer or the like, and then the molded body is removed from the mold. In cases where an unreacted component remains after the removal, the unreacted component is preferably removed by washing with distilled water, ethanol, or the like. By this, an OCP molded body that is a composite containing the biocompatible polymer can be produced.

### [3] Third Embodiment

A third embodiment is a method of producing an OCP molded body that is a composite containing a biocompatible polymer, the method comprising the steps of:
mixing a compound containing a calcium ion with a solution containing a biocompatible polymer, to prepare a mixed slime;
filling the mixed slime into a mold, and then freezing the mixed slime; and
immersing the molded body obtained by the freezing, in a solution of a compound containing a phosphate ion, to react the calcium ion with the phosphate ion.

In the third embodiment, calcium phosphate (excluding OCP) may be used as the compound containing a calcium ion. Examples of the calcium phosphate include calcium dihydrogen phosphate hydrate, anhydrous calcium dihydrogen phosphate, calcium hydrogen phosphate dihydrate, anhydrous calcium hydrogen phosphate, hydroxyapatite, carbonate apatite, sodium-substituted hydroxyapatite, α-tricalcium phosphate, and β-tricalcium phosphate.

Besides the calcium phosphates described above, inorganic calcium salts or organic calcium salts may be used as the compound containing a calcium ion. Examples of the inorganic calcium salts include calcium carbonate, calcium hydroxide, calcium oxide, calcium chloride, calcium fluoride, calcium bromide, calcium iodide, calcium phosphide, calcium sulfate, calcium sulfate hemihydrate, calcium sulfate dihydrate, anhydrous calcium oxalate, calcium oxalate monohydrate, calcium oxalate dihydrate, calcium oxalate trihydrate, calcium sulfite, calcium silicate, calcium pyrophosphate, calcium tungstate, and calcium molybdate. Examples of the organic calcium salts include calcium acetate, calcium succinate, calcium citrate, calcium malate, calcium thiomalate, calcium benzoate, calcium lactate, and calcium stearate. These may be used either individually or as a mixture containing two or more thereof at any ratios.

In the third embodiment, as the solution of a compound containing a phosphate ion, a solution containing an inorganic salt of phosphoric acid may be used. Examples of the inorganic salt of phosphoric acid include sodium dihydrogen phosphate, disodium hydrogen phosphate, trisodium phosphate, potassium dihydrogen phosphate, dipotassium hydrogen phosphate, tripotassium phosphate, ammonium dihydrogen phosphate, diammonium hydrogen phosphate, triammonium phosphate, and ammonium magnesium phosphate. These may be used either individually or as a mixture containing two or more thereof at any ratios.

In the third embodiment, a step of mixing a compound containing a calcium ion with a solution containing a biocompatible polymer, to prepare a mixed slime, is followed by filling the mixed slime into a mold. For example, a raw material calcium carbonate powder is provided, and a solution containing a biocompatible polymer obtained by dissolving the biocompatible polymer in a solvent is mixed with the raw material powder, to prepare a mixed slime. The mixing ratio is preferably 200:1 to 1:1 in terms of the weight ratio between the compound containing a calcium ion, and the biocompatible polymer. The mixing ratio is more preferably 100:1 to 2:1, still more preferably 50:1 to 3:1, especially preferably 20:1 to 5:1.

Subsequently, the mixed slime is filled into a mold, and then frozen. The freezing temperature is preferably not more than −15° C. Although there is no lower limit of the freezing temperature, the freezing temperature is preferably not less than-50° C. After the freezing, the mixed slime is dried (freeze-dried) in the frozen state, to remove water. The frozen (preferably freeze-dried) molded body is removed from the mold, and then immediately immersed in a solution of a compound containing a phosphate ion. By this, the calcium ion can be reacted with the phosphate ion, and the state of the biocompatible polymer can be changed to achieve insolubilization and maintenance of the shape. The immersion temperature and the immersion time may be appropriately set. The immersion temperature may be, for example, 50 to 70° C. The immersion time may be, for example, 10 minutes to 96 hours. During this immersion process, the frozen molded body thaws, but, in parallel, calcium phosphate crystals containing the biocompatible polymer precipitate to maintain the shape. By this, an OCP molded body that is a composite containing the biocompatible polymer can be produced.

### (OCP Molded Body)

The molded bodies obtained by the first to third embodiments can achieve a DTS strength of not less than 0.1 MPa, not less than 0.3 MPa, or not less than 0.5 MPa. In cases where the DTS strength is not less than 0.1 MPa, the molded body can be pinched rather strongly with tweezers without causing disintegration. Therefore, the molded body can be easily implanted into a bone defect site in vivo. From the viewpoint of allowing easier operation, the molded body preferably has a higher strength.

The shape of the molded body basically depends on the shape of the mold into which the mixed slime is filled. Since the molded body has a sufficient strength, it can also be processed into a particular shape. For example, the molded body can be processed into a cube, a rectangular parallelepiped, a cylinder, a cone, a conical trapezoid, a sphere, an octahedron, a tetrahedron, or the like. The molded body can have a volume of not less than 1 mm³, not less than 3 mm³, or, in particular, not less than 5 mm³. On the other hand, a volume of more than 1000 mm³ may result in reduction of the strength.

### Examples

A description is given below by way of Examples. The present Examples are merely examples, and the present invention is not limited by these Examples. This means that the present invention is limited only by claims, and that the present invention encompasses various modifications other than the Examples included in the present invention.

Physical properties of each molded body were evaluated using the following measurement devices and measurement conditions.

### (DTS Strength)

The diametral tensile strength (DTS strength) was measured using a universal tester AGS-J, manufactured by Shimadzu Corporation, at a head speed of 1 mm/min.

### (X-Ray Diffraction Analysis)

XRD analysis was carried out using an X-ray diffraction analyzer (MiniFlex 600, Rigaku Corporation, Japan; target, Cu; wavelength, 0.15406 nm). Regarding the XRD measurement conditions, the acceleration voltage and the amplitude were 40 kV and 15 mA, respectively. Properties were evaluated by continuous scanning of the diffraction angle for the value of 20 from 3° to 70° at an operating speed of 2°/minute. When the molded body was analyzed by XRD, formation of OCP was confirmed by evaluating the peak of OCP as well as the peak of the biocompatible polymer.

### (Stress-Strain Curve)

A stress-strain curve was obtained by performing measurement using a universal tester AGS-J, manufactured by Shimadzu Corporation, at a head speed of 1 mm/min.

### (Electron Microscopy)

A microstructure of each sample was evaluated using a field-emission-type scanning electron microscope (FE-SEM: JSM-6700F, JEOL Ltd., Japan). The acceleration voltage was set to 5 kV. In order to prevent the surface charge accumulation, the sample was subjected to sputter coating with Os.

### (FT-IR Analysis)

Properties of the chemical oscillation scheme of each sample were evaluated by Fourier transform infrared spectroscopy (FT-IR: Nicolet NEXUS670, Thermo Fisher Scientific Inc., USA) using a triglycine sulfate detector equipped with an attenuated total reflection prism made of GeSe (32 scans; resolution, 2 cm⁻¹). The atmosphere was taken into account as the background for carrying out the measurement.

### (Micro-CT Analysis)

An internal microstructure of each sample was evaluated using a micro-CT analyzer (micro-CT: Skyscan 1085, TOYO Corporation). The acceleration voltage was set to 60 kV. The evaluation was carried out using an A1 filter, with a resolution of 9μ m.

### (Example 1) Alginic Acid-OCP Molded Body

In an automatic agate mortar, 2.40 g of calcium hydrogen phosphate dihydrate (DCPD) and 2.84 g of disodium hydrogen phosphate (NaAP) were mixed together for not less than 10 minutes at a speed of 100 rpm, to prepare a mixed powder. The mixed powder was placed in a polystyrene tray, and an aqueous ammonium alginate solution with a concentration of 50 g/L was added dropwise thereto at a water/powder ratio of 1.0. The resulting mixture was thoroughly mixed using a spatula, to prepare a mixed slime, and the mixed slime was filled into a silicone mold with a diameter of 6 mm and a thickness of 3 mm, followed by allowing the mixed slime to cure in a sealed state at 40° C. for 3 days. Thereafter, the mixed slime was air-dried at 40° C. for not less than 12 hours, and the dried mixed slime was removed from the mold. The mixed slime was then washed thoroughly with distilled water to remove remaining unreacted components, and then dried again at 40° C.

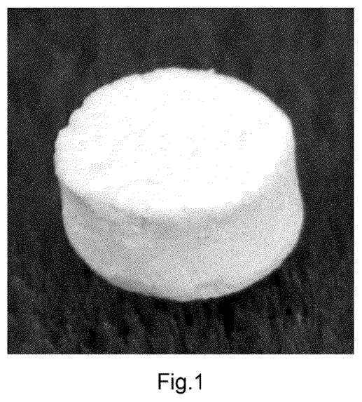

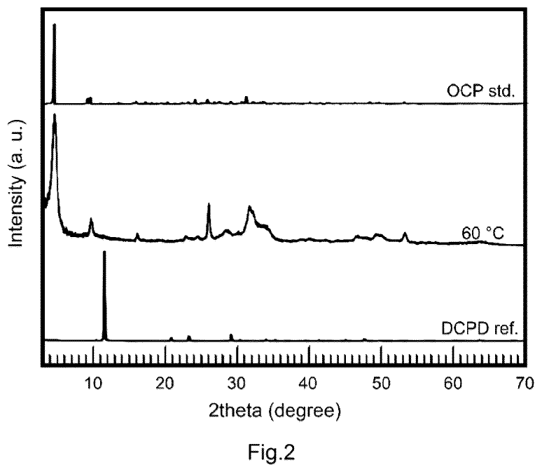

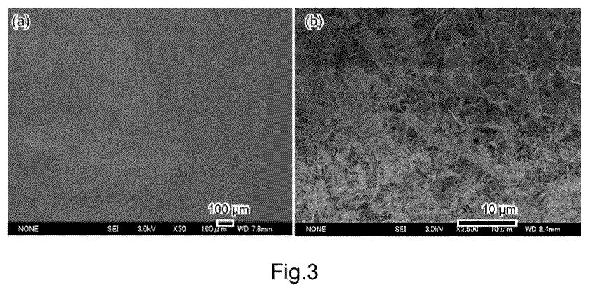

FIG. 1 shows a photograph of the molded body obtained. It can be seen from FIG. 1 that the molded body maintained the shape of the mold. The molded body had a DTS strength of 0.18±0.04 MPa, which corresponds to a strength at which the molded body can be pinched with tweezers without causing disintegration. FIG. 2 shows the result of XRD analysis of the molded body. From FIG. 2, the molded body obtained was found to be a single phase of OCP. A portion of the molded body was cut out, and its fracture surface was observed by FE-SEM. The observation result is shown in FIG. 3. The cross-section showed a dense accumulation of scale-like crystals each having a size of about several ten nanometers, in which the crystals were partially fused to each other. Further, some crystals had fibrous structures therebetween.

### (Example 2) Gelatin-OCP Molded Body

In an automatic agate mortar, 2.40 g of calcium hydrogen phosphate dihydrate (DCPD) and 2.84 g of disodium hydrogen phosphate (NaAP) were mixed together for not less than 10 minutes at a speed of 100 rpm, to prepare a mixed powder. The mixed powder was placed in a polystyrene tray, and an aqueous gelatin solution (concentration: 10, 50, or 100 g/L) was individually added dropwise to the mixed powder at a water/powder ratio of 1.0. The resulting mixture was thoroughly mixed using a spatula, to prepare a mixed slime, and the mixed slime was filled into a silicone mold with a diameter of 6 mm and a thickness of 3 mm, followed by allowing the mixed slime to cure in a sealed state at 40° C. for 3 days. Thereafter, the mixed slime was air-dried at 40° C. for not less than 12 hours, and the dried mixed slime was removed from the mold. The mixed slime was then washed thoroughly with distilled water to remove remaining unreacted components, and then dried again at 40° C.

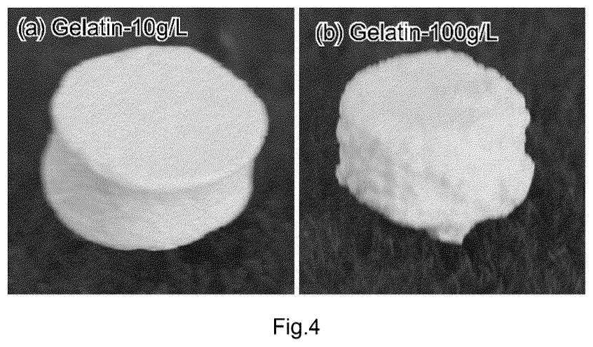

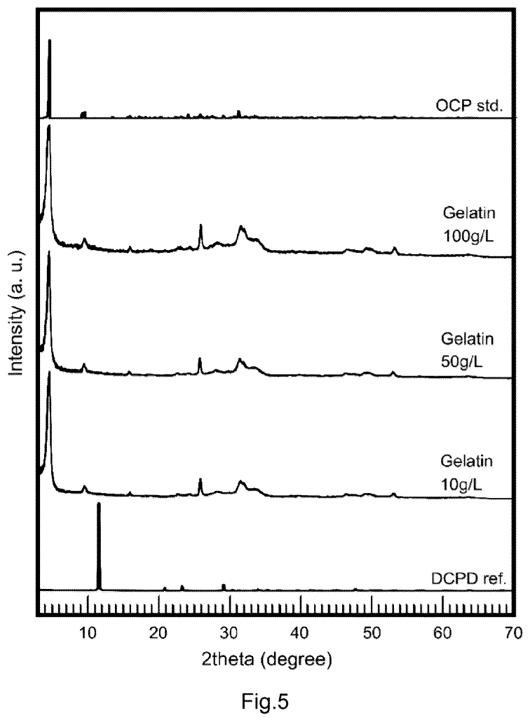

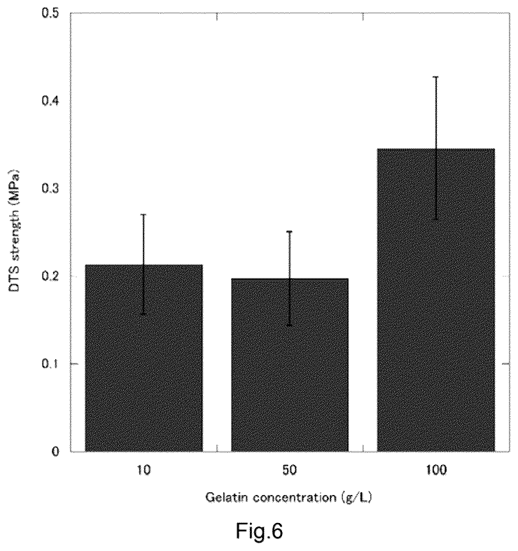

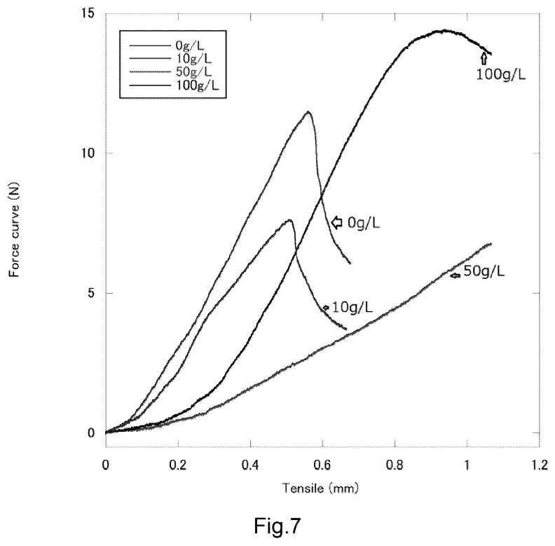

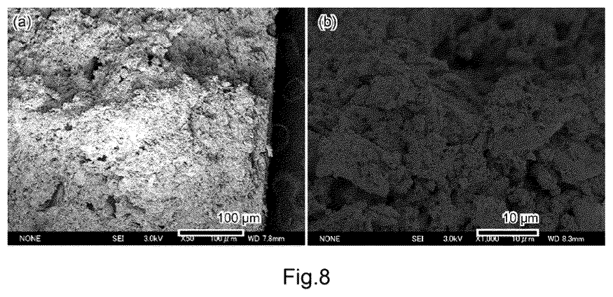

FIG. 4 shows photographs of molded bodies obtained. It can be seen from FIG. 4 that each molded body maintained the shape of the mold. FIG. 5 shows the result of XRD analysis of each molded body. From FIG. 5, it can be seen that each molded body obtained is a single phase of OCP. FIG. 6 shows the result of measurement of the DTS strength. Each molded body had a DTS strength of not less than 0.18±0.04 MPa, which corresponds to a strength at which the molded body can be pinched with tweezers without causing disintegration. FIG. 7 shows a stress-strain curve of each molded body, which indicates that each molded body does not disintegrate even by application of a certain level of strain. A portion of a molded body was cut out, and its fracture surface was observed by FE-SEM. The observation result is shown in FIG. 8. The cross-section showed a dense accumulation of scale-like crystals each having a size of about several ten nanometers, in which the crystals were partially fused to each other. Further, some crystals had fibrous structures therebetween.

### (Example 3) Sodium Polyacrylate-OCP Molded Body

In an automatic agate mortar, 2.40 g of calcium hydrogen phosphate dihydrate (DCPD) and 2.84 g of disodium hydrogen phosphate (NaAP) were mixed together for not less than 10 minutes at a speed of 100 rpm, to prepare a mixed powder. The mixed powder was placed in a polystyrene tray, and an aqueous sodium polyacrylate acid (PAA-Na) solution with a concentration of 100 g/L was added dropwise thereto at a water/powder ratio of 1.0. The resulting mixture was thoroughly mixed using a spatula, to prepare a mixed slime, and the mixed slime was filled into a silicone mold with a diameter of 6 mm and a thickness of 3 mm, followed by allowing the mixed slime to cure in a sealed state at 40° C. for 3 days. Thereafter, the mixed slime was air-dried at 40° C. for not less than 12 hours, and the dried mixed slime was removed from the mold.

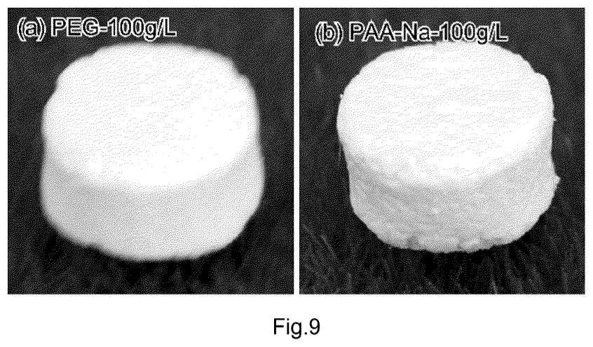

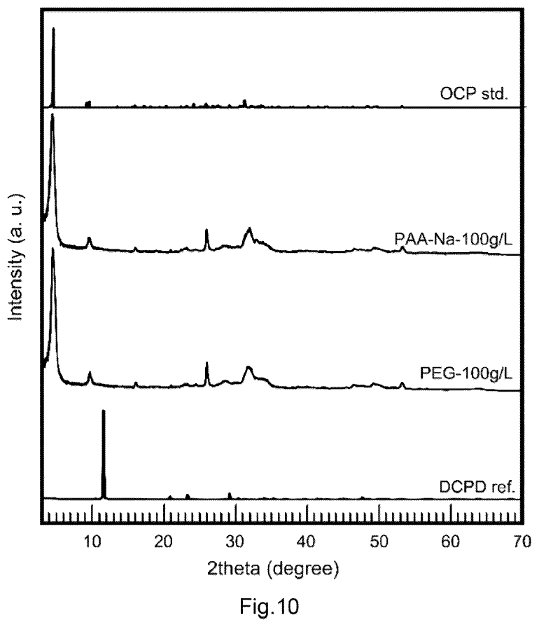

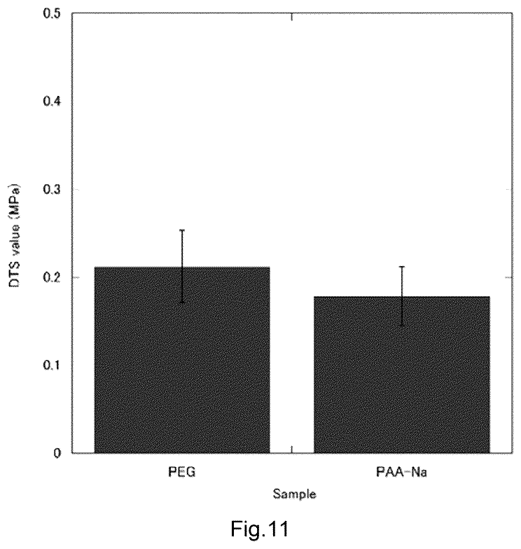

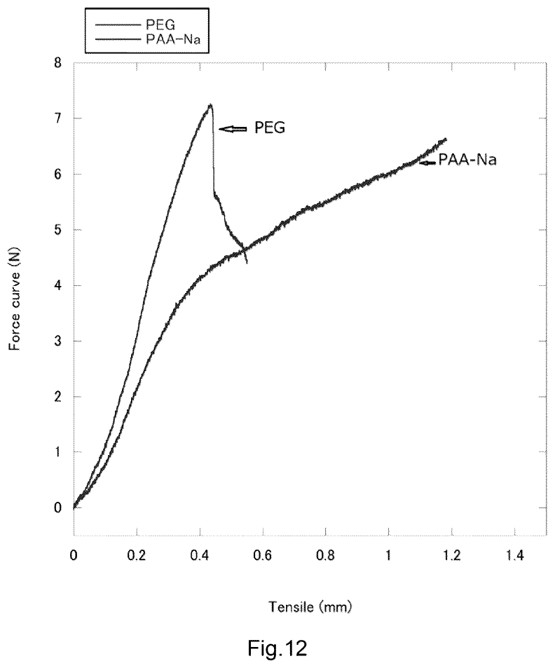

FIG. 9 (right panel) shows photographs of the molded body that is a composite containing PAA-Na. It can be seen from FIG. 9 (right panel) that the molded body maintained the shape of the mold. FIG. 10 shows the result of XRD analysis of the molded body (PAA-Na-100 g/L). From FIG. 10, it can be seen that the molded body obtained is a single phase of OCP. FIG. 11 shows the DTS strength of the molded body (PAA-Na). Each molded body had a DTS strength of not less than 0.18±0.04 MPa, which corresponds to a strength at which the molded body can be pinched with tweezers without causing disintegration. FIG. 12 shows a stress-strain curve of the molded body (PAA-Na). It can be seen that the molded body does not exhibit a significant breaking strength, and shows a high plastic deformation. In other words, the molded body was a soft molded body.

### (Example 4) Polyethylene Glycol-OCP Molded Body

In an automatic agate mortar, 2.40 g of calcium hydrogen phosphate dihydrate (DCPD) and 2.84 g of disodium hydrogen phosphate (NaAP) were mixed together for not less than 10 minutes at a speed of 100 rpm, to prepare a mixed powder. The mixed powder was placed in a polystyrene (PS) tray, and an aqueous polyethylene glycol (PEG) solution with a concentration of 100 g/L was individually added dropwise thereto at a water/powder ratio of 1.0. The resulting mixture was thoroughly mixed using a spatula, to prepare a mixed slime, and the mixed slime was filled into a silicone mold with a diameter of 6 mm and a thickness of 3 mm, followed by allowing the mixed slime to cure in a sealed state at 40° C. for 3 days. Thereafter, the mixed slime was air-dried at 40° C. for not less than 12 hours, and the dried mixed slime was removed from the mold.

FIG. 9 (left panel) shows photographs of the molded body that is a composite containing PEG. It can be seen from FIG. 9 (left panel) that the molded body maintained the shape of the mold. FIG. 10 shows the result of XRD analysis of the molded body (PEG-100 g/L). From FIG. 10, it can be seen that the molded body obtained is a single phase of OCP. FIG. 11 shows the DTS strength of the molded body (PEG). Each molded body had a DTS strength of not less than 0.18±0.04 MPa, which corresponds to a strength at which the molded body can be pinched with tweezers without causing disintegration. FIG. 12 shows a stress-strain curve of the molded body (PEG). It can be seen that the molded body does not exhibit a significant breaking strength, and shows high plastic deformation. In other words, the molded body was a soft molded body.

### (Example 5) Gelatin-OCP Molded Body

In an agate mortar, 1.75 g of calcium carbonate was placed. An aqueous gelatin solution (concentration: 10 g/L or 100 g/L) was individually added dropwise thereto at a water/powder ratio of 1.0, and the resulting mixture was thoroughly mixed to prepare a slurry. To this slurry, 2 mL of 4 mol/L aqueous phosphoric acid solution was added dropwise, and the resulting mixture was quickly stirred with a pestle, to prepare a mixed slime. During the mixing, foaming occurred vigorously, and partial curing occurred. The mixing was continued until this process settled down. The mixed slime was filled into a silicone mold with a diameter of 6 mm and a thickness of 3 mm, followed by allowing the mixed slime to cure in a sealed state at 40° C. for 3 days. Thereafter, the mixed slime was air-dried at 40° C. for not less than 12 hours, and the dried mixed slime was removed from the mold. Subsequently, the mixed slime was washed with distilled water, and then dried.

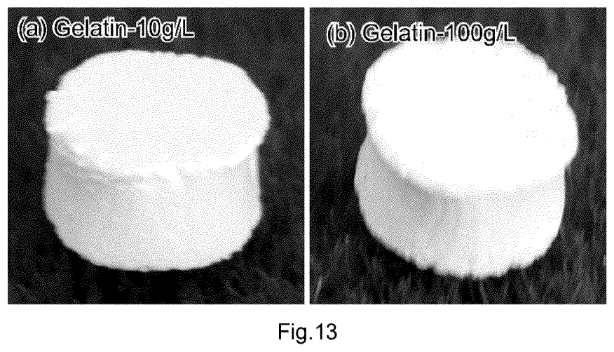

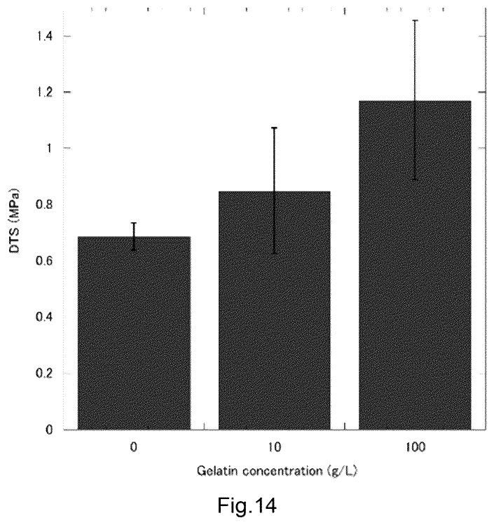

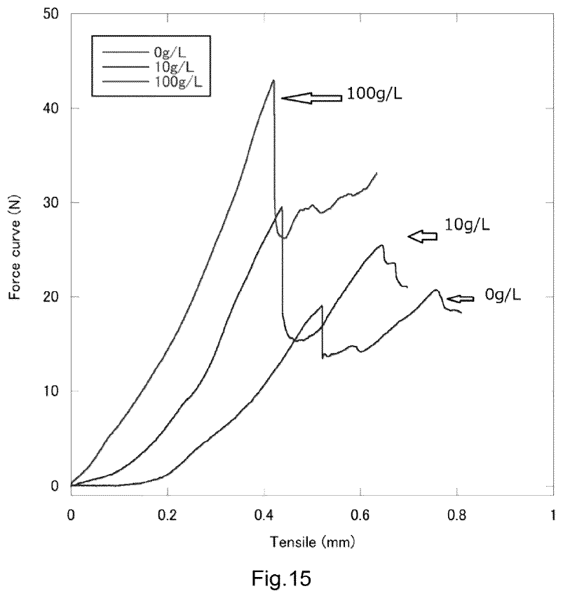

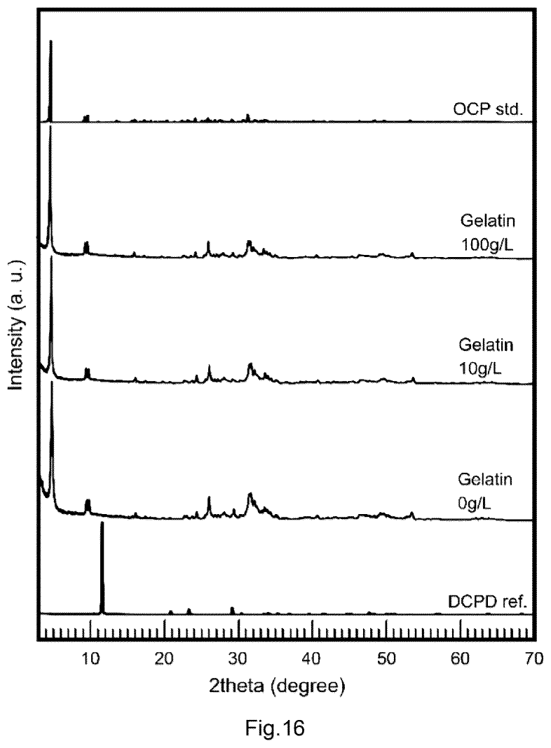

FIG. 13 shows photographs of the molded bodies obtained. It can be seen from FIG. 13 that each molded body maintained the shape of the mold. FIG. 14 shows the DTS strength of each molded body. Each molded body had a DTS strength of not less than 0.18±0.04 MPa, which corresponds to a strength at which the molded body can be pinched with tweezers without causing disintegration. FIG. 15 shows a stress-strain curve of each molded body. It can be seen that each molded body does not exhibit a significant breaking strength, and shows high plastic deformation. FIG. 16 shows the result of XRD analysis of each molded body. From FIG. 16, each molded body obtained was found to be a single phase of OCP.

### (Example 6) Sodium Polyacrylate-OCP Molded Body

In an agate mortar, 1.75 g of calcium carbonate was placed. Subsequently, 1 mL of an aqueous sodium polyacrylate acid (PAA-Na) solution with a concentration of 10 g/L was added dropwise thereto, and the resulting mixture was thoroughly mixed to prepare a slurry. To this slurry, 2 mL of 4 mol/L aqueous phosphoric acid solution was added dropwise, and the resulting mixture was quickly stirred with a pestle, to prepare a mixed slime. During the mixing, foaming occurred vigorously, and partial curing occurred. The mixing was continued until this process settled down. The mixed slime was filled into a silicone mold with a diameter of 6 mm and a thickness of 3 mm, followed by allowing the mixed slime to cure in a sealed state at 40° C. for 3 days. Thereafter, the mixed slime was dried at 40° C. for not less than 12 hours, and the dried mixed slime was removed from the mold. Subsequently, the mixed slime was washed with distilled water, and then dried.

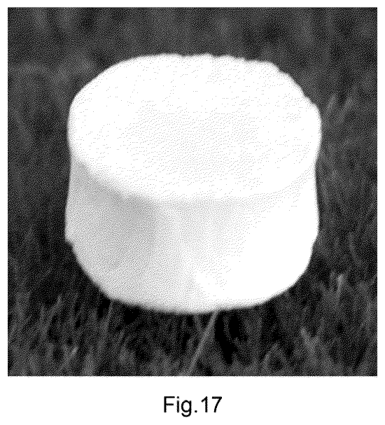

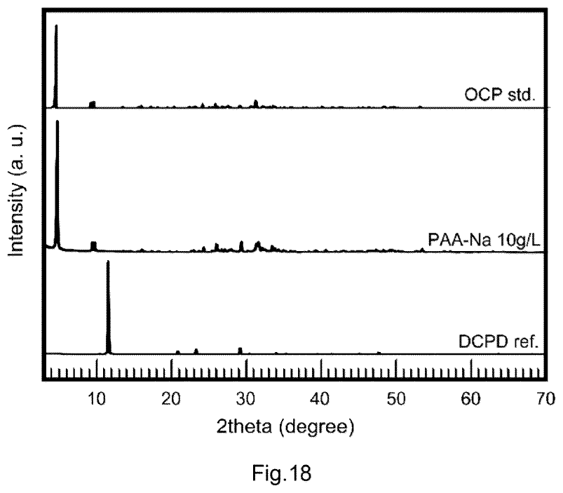

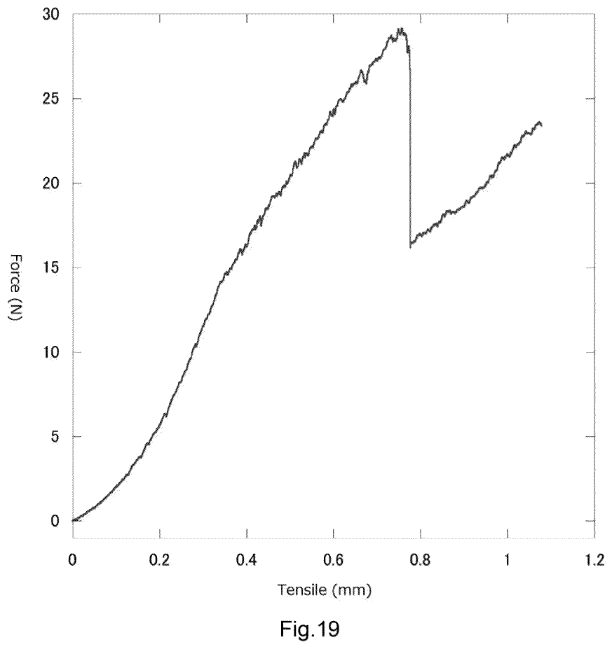

FIG. 17 shows a photograph of the molded body obtained. It can be seen from FIG. 17 that the molded body maintained the shape of the mold. FIG. 18 shows the XRD pattern of the molded body, and FIG. 19 shows the DTS strength of the molded body. The molded body had a DTS strength of not less than 0.88±0.04 MPa, which corresponds to a strength at which the molded body can be pinched with tweezers without causing disintegration.

### (Example 7) Alginic Acid-OCP Molded Body

A mixed slime was prepared by mixing 1 g of calcium hydrogen phosphate dihydrate (DCPD) with an aqueous ammonium alginate solution with a concentration of 50 g/L at a water/powder ratio of 1.0. The mixed slime was filled into a silicone mold with a diameter of 6 mm and a thickness of 3 mm, and then frozen at −20° C., followed by drying the mixed slime by evaporating the water in a freeze dryer. The dried molded body maintained the shape of the mold. Thereafter, the molded body was immersed in 20 mL of an aqueous disodium hydrogen phosphate (NaAP) solution with a concentration of 1 mol/L, and then the reaction was allowed to proceed at 60° C. for 1 day. During the reaction, the shape of the molded body was maintained even though foaming occurred therefrom.

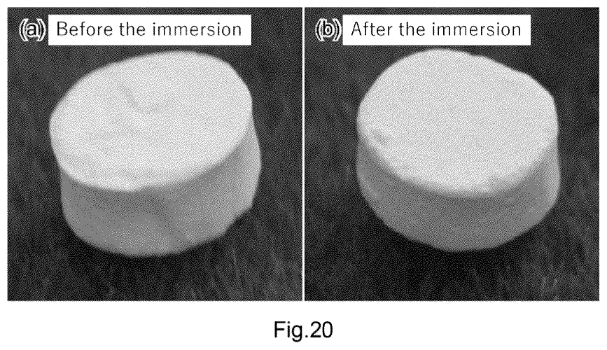

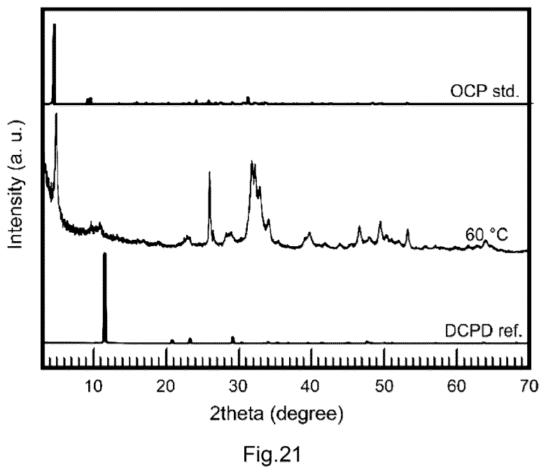

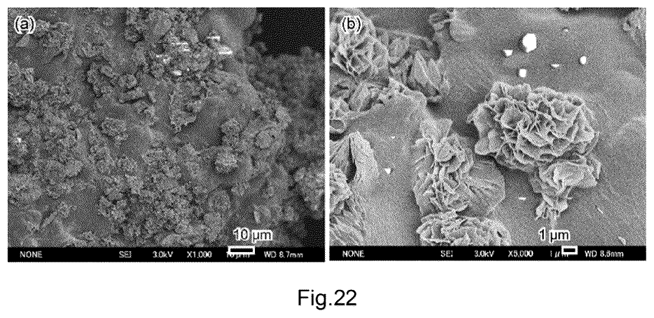

FIG. 20 shows photographs of the molded body taken before and after the immersion. It can be seen from FIG. 20 that the molded body maintained the shape of the mold even after the immersion. Pores of several hundred micrometers were found due to the formation of ice crystals by the freezing. The molded body had a DTS strength of not less than 0.54±0.21 MPa. FIG. 21 shows the result of XRD analysis of the molded body after the immersion. As can be seen in FIG. 21, the molded body obtained showed partial formation of OCP. FIG. 22 shows SEM images of the molded body after the immersion. Radial OCP crystals were found to be embedded in alginic acid fibers.

### (Example 8) Alginic Acid-OCP Molded Body

A mixed slime was prepared by mixing 1 g of calcium carbonate with an aqueous ammonium alginate solution with a concentration of 50 g/L at a water/powder ratio of 1.0. The mixed slime was filled into a silicone mold with a diameter of 6 mm and a thickness of 3 mm, and then frozen at −20° C., followed by drying the mixed slime by evaporating the water in a freeze dryer. The dried molded body maintained the shape of the mold. Thereafter, the molded body was immersed in 20 mL of phosphoric acid with a concentration of 100 mol/L, and then the reaction was allowed to proceed at 60° C. for 3 days. During the reaction, the shape of the molded body was maintained even though foaming occurred therefrom.

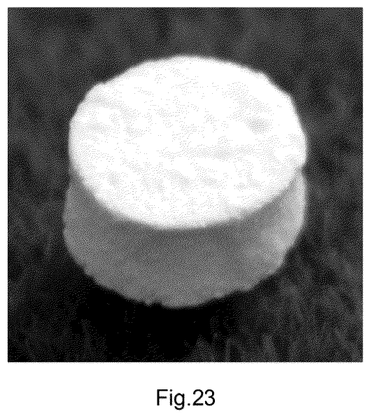

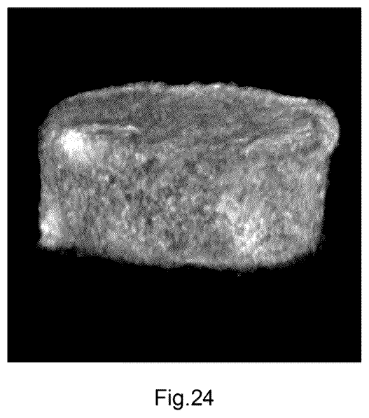

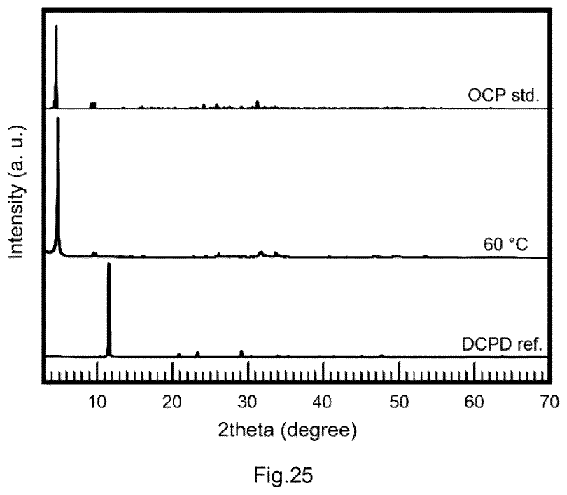

FIG. 23 shows photographs of the molded body taken after the immersion. It can be seen from FIG. 23 that the molded body maintained the shape of the mold even after the immersion. FIG. 24 shows a CT image of the molded body. Pores of several hundred micrometers were found due to the formation of ice crystals by the freezing. The molded body had a DTS strength of 0.32±0.12 MPa. FIG. 25 shows the result of XRD analysis of the molded body after the immersion. As can be seen in FIG. 25, the molded body obtained showed partial formation of octacalcium phosphate (OCP).

1-6. (canceled)

7. A method of producing an octacalcium phosphate molded body that is a composite containing a biocompatible polymer, the method comprising the steps of:
mixing a compound containing a calcium ion with a solution containing a biocompatible polymer, to prepare a mixed slime; and
causing the mixed slime or a molded body thereof to interact with a compound containing a phosphate ion to react the calcium ion with the phosphate ion.

8. The method of producing an octacalcium phosphate molded body that is a composite containing a biocompatible polymer according to claim 7, wherein
the mixing comprises mixing the compound containing the calcium ion and the compound containing the phosphate ion with the solution containing the biocompatible polymer, to prepare the mixed slime; and
the causing comprises filling the mixed slime into a mold, to react the calcium ion with the phosphate ion.

9. The method of producing an octacalcium phosphate molded body that is a composite containing a biocompatible polymer according to claim 7, wherein
the causing comprises:
adding a solution of the compound containing the phosphate ion to the mixed slime, and mixing a resulting mixture; and
filling the resulting mixture obtained after the mixing into a mold, to react the calcium ion with the phosphate ion.

10. The method of producing an octacalcium phosphate molded body that is a composite containing a biocompatible polymer according to claim 7, further comprising:
filling the mixed slime into a mold, and then freezing the mixed slime to form the molded body, wherein
the causing comprises immersing the molded body obtained by the freezing, in a solution of the compound containing the phosphate ion, to react the calcium ion with the phosphate ion.

11. The method of producing an octacalcium phosphate molded body that is a composite containing a biocompatible polymer according to claim 8, wherein the compound containing a calcium ion is one or more selected from the group consisting of calcium dihydrogen phosphate hydrate, anhydrous calcium dihydrogen phosphate, calcium hydrogen phosphate dihydrate, anhydrous calcium hydrogen phosphate, hydroxyapatite, carbonate apatite, sodium-substituted hydroxyapatite, α-tricalcium phosphate, β-tricalcium phosphate, and calcium carbonate.

12. The method of producing an octacalcium phosphate molded body that is a composite containing a biocompatible polymer according to claim 8, wherein the compound containing a phosphate ion is one or more selected from the group consisting of sodium dihydrogen phosphate, disodium hydrogen phosphate, trisodium phosphate, potassium dihydrogen phosphate, dipotassium hydrogen phosphate, tripotassium phosphate, ammonium dihydrogen phosphate, diammonium hydrogen phosphate, triammonium phosphate, and ammonium magnesium phosphate.

13. The method of producing an octacalcium phosphate molded body that is a composite containing a biocompatible polymer according to claim 8, wherein the biocompatible polymer is one or more of alginate, gelatin, hyaluronic acid, gellan gum, carboxylic acid-modified cellulose, sodium polyacrylate acid, polyethylene glycol, pullulan, phosphate modified pullulan, polyglutamic acid, fibrin, chitin, chitosan, and gellan gum.

14. The method of producing an octacalcium phosphate molded body that is a composite containing a biocompatible polymer according to claim 9, wherein the compound containing a calcium ion is one or more selected from the group consisting of calcium dihydrogen phosphate hydrate, anhydrous calcium dihydrogen phosphate, calcium hydrogen phosphate dihydrate, anhydrous calcium hydrogen phosphate, hydroxyapatite, carbonate apatite, sodium-substituted hydroxyapatite, α-tricalcium phosphate, β-tricalcium phosphate, and calcium carbonate.

15. The method of producing an octacalcium phosphate molded body that is a composite containing a biocompatible polymer according to claim 9, wherein the compound containing a phosphate ion is one or more selected from the group consisting of sodium dihydrogen phosphate, disodium hydrogen phosphate, trisodium phosphate, potassium dihydrogen phosphate, dipotassium hydrogen phosphate, tripotassium phosphate, ammonium dihydrogen phosphate, diammonium hydrogen phosphate, triammonium phosphate, and ammonium magnesium phosphate.

16. The method of producing an octacalcium phosphate molded body that is a composite containing a biocompatible polymer according to claim 9, wherein the biocompatible polymer is one or more of alginate, gelatin, hyaluronic acid, carboxylic acid-modified cellulose, sodium polyacrylate acid, polyethylene glycol, pullulan, phosphate modified pullulan, polyglutamic acid, fibrin, chitin, chitosan, and gellan gum.

17. The method of producing an octacalcium phosphate molded body that is a composite containing a biocompatible polymer according to claim 10, wherein the compound containing a calcium ion is one or more selected from the group consisting of calcium dihydrogen phosphate hydrate, anhydrous calcium dihydrogen phosphate, calcium hydrogen phosphate dihydrate, anhydrous calcium hydrogen phosphate, hydroxyapatite, carbonate apatite, sodium-substituted hydroxyapatite, α-tricalcium phosphate, β-tricalcium phosphate, and calcium carbonate.

18. The method of producing an octacalcium phosphate molded body that is a composite containing a biocompatible polymer according to claim 10, wherein the compound containing a phosphate ion is one or more selected from the group consisting of sodium dihydrogen phosphate, disodium hydrogen phosphate, trisodium phosphate, potassium dihydrogen phosphate, dipotassium hydrogen phosphate, tripotassium phosphate, ammonium dihydrogen phosphate, diammonium hydrogen phosphate, triammonium phosphate, and ammonium magnesium phosphate.

19. The method of producing an octacalcium phosphate molded body that is a composite containing a biocompatible polymer according to claim 10, wherein the biocompatible polymer is one or more of alginate, gelatin, hyaluronic acid, carboxylic acid-modified cellulose, sodium polyacrylate acid, polyethylene glycol, pullulan, phosphate modified pullulan, polyglutamic acid, fibrin, chitin, chitosan, and gellan gum.

20. The method of producing an octacalcium phosphate molded body that is a composite containing a biocompatible polymer according to claim 9, wherein the causing further comprises curing the resulting mixture filled into the mold at 35 to 50° C. to react the calcium ion with phosphate ion.
